(19) United States
(12) Patent Application Publication
(10) Pub. No.: US 2005/0208670 A1
(43) Pub. Date: Sep. 22, 2005

(54) Detection of mercury in biological samples

(76) Inventors: Malcolm B. Wittenberg, San Rafael, CA (US); Travis Honeycutt, Gainesville, GA (US); Viktor Sharivker, Ottawa (CA); Vladimir Blinov, Ottawa (CA); Simon Sharivker, Ottawa (CA)

Correspondence Address: Malcolm B. Wittenberg, 160 Mitchell Boulevard, San Rafael, CA, 94903, US

(21) Appl. No.: 10/805,599

(22) Filed: Mar. 22, 2004

(51) Int. Cl. G01N 33/00

(52) U.S. Cl. 436/81; 422/50; 422/68.1

(57) ABSTRACT

A device and method for determining mercury levels in a biological sample containing mercury. The biological sample is at least partially dissolved in an acidic solution to release at least a portion of the mercury contained therein. The partially dissolved biological sample is exposed to an anode and cathode under an electromotive force causing at least a portion of the mercury released from the biological sample to adhere to a surface of the cathode. The cathode being exposed to an alkaline metal salt to create a mercury-alkaline metal amalgam. This cathode is then electrically connected to a reference electrode whereby the extent of the amalgam is a measure of the level of mercury in the biological sample.

### TECHNICAL FIELD

The present invention involves a simple to use and highly accurate method and device for determining concentrations of mercury, a highly toxic naturally occurring element, in biological matter such as fish.

### BACKGROUND OF THE INVENTION

Mercury, as a naturally occurring element, vaporizes in the air and leaches into rivers, lakes and oceans. Plant life, animals and fish consume mercury-containing bacteria and store mercury, generally as methyl mercury, its most toxic state, in various levels of concentration.

Although a wide variety of maladies have been attributed to mercury, medical researchers generally are in agreement that high levels of mercury can cause brain damage, infertility, and, in extreme cases, even death. The EPA warns that high level exposure to methyl mercury can impair central nervous system function, cause kidney, gastrointestinal, cardiovascular and immune system damage, and even lead to shock or death. The FDA warns pregnant women to avoid eating shark, swordfish, king mackerel and tilefish. The FDA also warns nursing mothers and young children as methyl mercury can damage nervous systems in the unborn and young.

Consumers are growing increasingly anxious about the possibility of ingesting methyl mercury when they consume biological material, particularly seafood. Ironically, many health-conscious consumers have reduced their red meat intake as reports were circulated of the ill effects of even moderate red meat consumption, due to the presence of growth hormones in most commercially available beef and the high saturated fat levels of beef generally. Many dietary experts have suggested seafood consumption as a healthier alternative to red meat. As a result, the most health conscious segment of our population began increasing seafood intake only to discover that the same “healthy” alternative may contain unsafe levels of mercury.

Based upon existing data, the FDA has set a one part per million level as the maximum safe concentration level for mercury in fish. Other agencies have weighed in on this issue. For example, California has brought suit against five of the largest grocery chains which operate in that state as well as 20 restaurant chains forcing the grocery chains and restaurants to post warning labels at deli counters and on signage that at least some of the food being offered for sale can pose a health hazard thus forcing compliance with California's Prop 65, the 1986 voter-approved initiative that requires businesses to notify customers if they are being exposed to toxic chemicals.

Because of the above-noted litigation as well as a general awareness of the insidious effects that mercury can have, particularly upon young children and the unborn, there is a desire on the part of consumers as well as grocery chains and restaurants to test food being vended to confirm that such food does not contain excessive levels of mercury and to preferably indicate the mercury content of food being sold for human consumption. Unfortunately, prior to the present invention, there has been no consumer friendly, low cost means to test biological matter for mercury contamination.

Thus, it is an object of the present invention to provide an automated low cost device for enabling consumers and vendors to self test seafood in order to accurately determine mercury levels found therein.

This and further objects will be more readily apparent when considering the following disclosure and appended drawings.

### SUMMARY OF THE INVENTION

The present invention is directed to a method of determining mercury levels in a biological sample containing mercury as well as a device and kit for carrying out the claimed method. A biological sample containing mercury is at least partially dissolved in an acidic solution to release at least a portion of the mercury contained therein. The acid solution containing the mercury released from the biological sample is exposed to an anode and a cathode connected by an electromotive force causing at least a portion of the mercury released from the biological sample to adhere to a portion of the cathode surface. An alkaline metal salt solution can be incorporated into said acid solution or a second anode is exposed together with the cathode to an alkaline metal salt solution in a separate chamber under the influence of a second electromotive force established between the second anode and cathode. In either embodiment, an alkaline metal amalgam is formed on the surface of the cathode. After the amalgam has been created, the cathode is connected to a reference electrode and the voltage difference between the cathode and reference electrode is measured as an indicator of the mercury level in the biological sample.

### BRIEF DESCRIPTION OF THE FIGURES

FIG. 1 is a schematic representation in cross section of a first embodiment of the device of the present invention capable of carrying out the claimed method.

FIG. 2 is a schematic representation in cross section of a second embodiment of the device of the present invention capable of carrying out the claimed method.

### DETAILED DESCRIPTION OF THE INVENTION

The present device and method are capable of detecting fractions of a part per million of mercury in a biological sample. In fish, mercury is generally in the form of methyl mercury, that is, in its organic-bound form. As such, in order to accurately determine mercury levels, the biological matter must be at least partially decomposed to release the subject organic-bound metal.

It is recognized that a well accepted means of releasing metals from biological matter is through decomposition by incineration. However, in this instance, methyl mercury would tend to vaporize during incineration unless incineration conditions were strictly and carefully monitored. As such, incineration was not believed to be of practical value.

It has also been suggested that mercury in biological matter can be detected by the spectrographic analysis of an arc emission spectrum. However, spectrographic detection is sensitive to the presence of most other metals as well which would cause interfering conditions if one was to only seek an indicator of mercury.

Potentially, mercury concentration can also be determined by chemical methods whereby mercury (I) and/or (II) ions can be caused to react with certain reagents to provide colored compounds. However, it is often times difficult to differentiate between levels of such compounds in the fraction of parts per million concentrations such that distinguishing between the color and intensity of differing samples can prove to be a daunting experience. These various prior art bound limitations have effectively been overcome in practicing the present invention, described as follows.

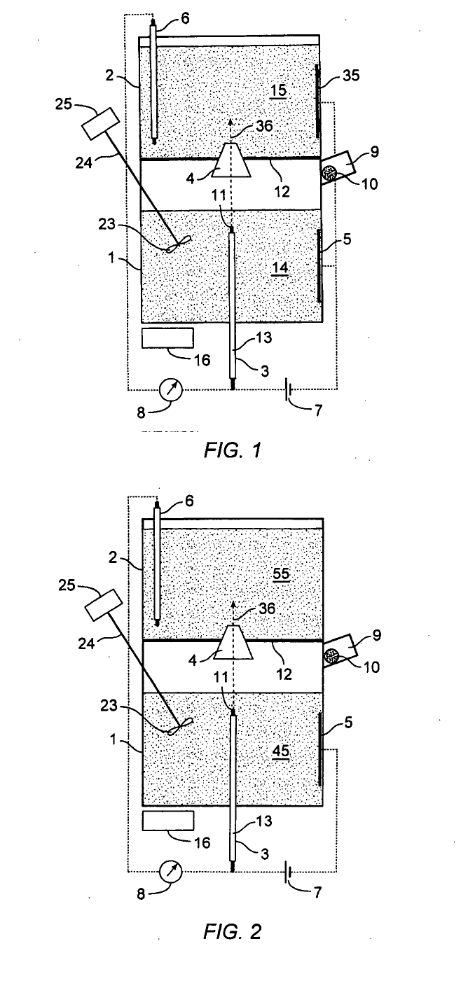

The present invention can best be understood with reference to FIGS. 1 and 2. Turning first to the embodiment depicted in FIG. 1, mercury detection cell 10 can be comprised of two chambers 1 and 2 separated by membrane 12 which can, as a preferred embodiment, comprise a septum-like self-sealing barrier. This barrier can be used alone or can be used together with a closable fitting 4 which can comprise, for example, a one way flapper valve whose use will be more readily appreciated in considering the discussion which follows.

In operation, cathode 3 is provided as including insulating coating 13 which covers the entire cathode except for exposed metallic tip proportion 11. In providing cathode 3 in this fashion, the exposed surface area of metallic tip portion 11 can be reproducibly established which greatly enhances reproducibility of the sought after mercury concentration results. Cathode 3 can be composed of one or more metals such as aluminum, platinum, gold, silver, zinc and copper. Optimally, electrode 3 is composed of aluminum.

A measured quantity of biological material, such as the fleshy portion of a fish body is inserted through port 9 and into chamber 1 containing a measured quantity of acidic solution 14. The quantity of the fish sample, generally from 0.5 to 5 gms, is provided for introduction within a measured quantity of acidic solution 14, generally from 0.5 to 5 mls, although larger samples can be measured using the present invention with corresponding larger quantities of acid and other reagents. As soon as the biological sample is introduced to acidic solution 14, decomposition begins thus releasing mercury ions from the intestacies of the biological matter. This step of the process can be carried out while stirring or mechanically or ultrasonically vibrating the solution to enhance uniformity. As such, stirrer 23 appended to motor 25 through shaft 24 can be employed or, alternatively vibrating motor or ultrasonic generator 16 can be used. It is proposed that digestion of the biological matter be conducted in the acidic solution in the presence of a strong oxidizing agent. Solutions which are applicable for practicing the present invention include hydrochloric acid with potassium chlorate, hydrochloric acid with chlorine, nitric acid alone or with sulfuric acid or with hydrogen peroxide or with potassium permanganate or with ammonium persulfate. As a preferred embodiment, the present invention has been carried out employing 12M concentrated hydrochloric acid together with potassium chlorate as oxidizer 10. This oxidizer is employed in quantities of from 0.1 to 0.5 grams when employing test samples of the quantity previously recited.

Through experimentation, it has been determined that a 3 gm sample of fish matter must remain in a concentrated hydrochloric acid solution for at least 70 hours in order to liberate all of the mercury contained therein. However, partial extraction, that is, the extraction of approximately 44% of the total mercury within the sample, can be achieved within approximately 5 minutes of digestion. This is an important recognition for a device requiring 70 hours to achieve a result would have, at best, limited practical utility.

It is noted that during the digestion process, gaseous chlorine (Cl₂) is evolved in the presence of concentrated HCl and KClO₃ according to the following reaction:
KClO₃₊6 HCl→3Cl₂+KCl+3H₂O
In proceeding with the digestion process, it is noted that Hg₂Cl₂ and HgCl₂ are formed as chlorine oxidizes the subject organic mercury compounds. These inorganic compounds are soluble in acidic solution 14 with excess chloride ions (Cl⁻) resulting in the formation of complex ions, for example, HgCl₄⁻².

In carrying out the claimed method, mercury ions extracted from the biological matter and thus present in acidic solution 14 are deposited electrochemically on the surface of metallic tip portion 11 of cathode 3. This is accomplished electrochemically by applying an electromotive force between cathode 3 and anode 5. The anode can be composed of, for example, carbon or platinum and electrolysis carried out through the use of DC power source 7. DC power source 7 can be in the form of a dry cell battery creating approximately 1.5 to 6 volts at a current of approximately 5 to 150 mA. Alternatively a regulated DC power supply can be employed providing either a constant voltage or constant current, the later being preferred. First anode 5 can be applied to the inner side wall of digestion chamber 1 in the form of a rod or plate.

In the first embodiment of FIG. 1, after the biological matter has been adequately digested within chamber 1 and mercury ions electrochemically plated to the surface of metallic tip portion 11 of cathode 3, the cathode can be caused to mechanically move in the direction of arrow 36 through at least partially sealable membrane 12 and ideally through one way valve fitting 4.

In the first embodiment, contained within second chamber 2 is solution 15 which is a salt of alkaline metal such as NaCl and NaSO₄. Ideally, solution 15 can comprise a buffered solution of sodium chloride caused to contact metallic tip portion 11 of cathode 3 although the alkaline metal could be any one of the members selected from the group consisting of potassium, sodium, lithium, rubidium and cesium. When this is carried out, cathode 3 can be electrically connected to second anode 35, again, constructed in the form of a rod or plate of, for example, carbon or platinum. A second electromotive force is applied between cathode 3 and second anode 35 resulting in the creation of an alkaline metal amalgam, in this case, sodium. During this step of the process, a voltage from approximately 7 to 15 volts is applied through power source 7 at a current of approximately 50 to 200 mA. The alkaline metal solution could have a pH from 5 to 8 and preferably 7 and can be in the form of an NaOH-KH₂PO₄ solution.

As an alternative embodiment, reference is made to FIG. 2 wherein like structural elements are numbered as elements of FIG. 1. In this embodiment however, as mercury ions are being electrochemically coated onto tip 11 of cathode 3, they are coated in the presence of an acid/alkaline metal salt solution 45 now contained within digestion chamber 1. In doing so, only one anode 5 is required. The same electromotive force used for plating mercury ions onto cathode tip 11 can be used to form the alkaline metal/mercury amalgam.

Whether the embodiment of FIG. 1 or FIG. 2 is employed, cathode 3 is caused to move into chamber 2 in the direction of arrow 36. The cathode is electrically connected to reference electrode 6 across meter 8. Reference electrode 6 can also be carbon or a metal of, for example, aluminum, platinum, gold or silver which is free of any amalgam. As such, the amalgam coated tip 11 creates an electromotive force with reference electrode 6 which acts as an indicator of the amount of mercury in the biological matter and thus, coated upon tip 11. In the embodiment of FIG. 1, liquid 15 contains a solution of a salt of alkaline metal while in FIG. 2, liquid 55 is water or other inert material.

To summarize, mercury ions are first discharged and then form mercury spots on the surface of metallic tip portion 11 of cathode 3 during the period of time in which metallic tip portion 11 resides within digestion chamber 1. In doing so, the following reactions take place at the cathode and anode surfaces:

At the cathode:
Hg⁺²+2e⁻→Hg
Hg₂⁺²+2e⁻→2Hg
2H⁺+2e⁻→H₂

At the anode:
2Cl⁻−2e⁻→Cl₂
In creating the alkaline metal amalgam on the surface of metallic tip portion 11, it was recognized that an alkaline metal, such as sodium, thermodynamically cannot be deposited on the metal (aluminum) electrode surface if mercury is not already present on such surface. As such, the amount of alkaline metal electrochemically adhering to the cathode surface formed in chamber 2 (FIG. 1) or chamber 1 (FIG. 2) is directly related to the amount of mercury electrochemically applied to such surface during the digestion step of the present invention. The creation of a sodium amalgam is electrochemically created pursuant to the following steps:

At the cathode:
2H⁺+2e⁻→H₂
Na⁺+Hg+e⁻→Na (in Hg)

At the anode:
2Cl⁻−2e⁻→Cl₂
2OH⁻−2e⁻→O₂+2H⁺
As a preferred embodiment, stability of the cathode could be enhanced by including a quantity of mercury ions in acidic solutions 14 or 45 to insure the creation of an alkaline metal amalgam in chamber 2 even if the biological sample was devoid of any mercury. In doing so any metering device used to reveal mercury content by measuring current between the cathode and reference electrode 6 would be adjusted to “zero” out the effects of the added mercury ions.

### EXAMPLE

Fish solutions which contain known amounts of mercury were used for calibration. A 2 g fish (salmon) sample which was known to contain no mercury was added to a plastic container which contained 0.2 g of solid KClO₃. 2 ml of 12 M HCl was then added into the mixture. A calibration solution of Hg(NO₃)₂ was then added after chlorine began evolving. Separate tests were conducted having known mercury concentrations of 0, 0.5, 1 and 2 micrograms that correspond to 0, 0.17, 0.33 and 0.67 ppm in the solution. Electrolysis of each solution was conducted using an aluminum cathode, carbon anode and batteries as a DC source. DC voltage was 4.5 V. Electrolysis time was 10 minutes. After electrolysis, electrodes were transferred into new cell for a second electrolysis. The new cell contained sodium chloride and buffer (pH=7) solution (NaOH—KH₂PO₄) as the electrolyte. Batteries were again used as the DC source. DC voltage was 7.5 V. Electrolysis time was 2 minutes. The aluminum cathode was then transferred into a separate cell which contained water and a reference electrode. Potentials of electrodes (cathode v. reference) were then measured. The cathode remained aluminum and the reference electrode was silver. The potential difference between electrodes in water was stable in limits of ±25 mV and representing a reliable indicator of the amount of mercury in the biological samples.

1. A method of determining mercury levels in a biological sample containing mercury comprising the steps of:
(a) at least partially dissolving said biological sample containing mercury in an acidic solution to release at least a portion of the mercury contained therein into said acidic solution;
(b) exposing said acidic solution to a first anode and a cathode connected by an electromotive force to adhere at least a portion of the mercury released from said biological sample to a surface of said cathode;
(c) providing a second anode and exposing said surface of said cathode and adhered mercury to a solution of an alkaline metal salt under the influence of an electromotive force established between said second anode and cathode to create an alkaline metal amalgam on the surface of said cathode;
(d) connecting said cathode to a reference electrode; and
(e) measuring any voltage difference between said cathode and reference electrode as a measure of the mercury level in the biological sample.

2. The method of claim 1 wherein said biological sample containing mercury comprises fish.

3. The method of claim 1 wherein said cathode comprises a member selected from the group consisting of aluminum, platinum, gold, silver, zinc and copper.

4. The method of claim 1 wherein said cathode comprises aluminum.

5. The method of claim 1 wherein said first and second anodes each comprise carbon or platinum.

6. The method of claim 1 wherein said reference electrode comprises a member selected from the group consisting of carbon, platinum, gold, silver and aluminum.

7. The method of claim 1 wherein an oxidizing agent is provided during the step of at least partially dissolving said biological sample containing mercury in said acidic solution.

8. The method of claim 7 wherein said oxidizing agent comprises potassium chlorate.

9. The method of claim 1 wherein said acid solution comprises an acid selected from the group consisting of hydrochloric acid, nitric acid, and sulfuric acid.

10. The method of claim 1 wherein said alkaline metal salt comprises a member selected from the group consisting of a salt of sodium, potassium, lithium, rubidium and cesium.

11. The method of claim 10 wherein said alkaline metal salt comprises a buffered solution of sodium chloride.

12. The method of claim 1 wherein said biological sample containing mercury comprises fish dissolved in a 12M hydrochloric acid solution in the presence of potassium chlorate as an oxidizer.

13. The method of claim 1 wherein said electromotive force of step (b) comprises a voltage between approximately 1.5 to 6 volts at a current of approximately 5 to 150 mA.

14. The method of claim 1 wherein said electromotive force of step (c) comprises a voltage between approximately 7 to 15 volts, at a current of approximately 50 to 200 mA.

15. The method of claim 1 wherein said electromatic force comprises a regulated power supply.

16. The method of claim 15 wherein said regulated power supply comprises a constant current power supply.

17. The method of claim 1 wherein said biological sample containing mercury is subjected to stirring or agitation while being dissolved in said acidic solution.

18. A method of determining mercury levels in a biological sample containing mercury comprising the steps of:
(a) at least partially dissolving said biological sample containing mercury in an acidic solution containing an alkaline metal salt to release at least a portion of the mercury contained therein into said acidic solution;
(b) exposing said acidic solution to an anode and a cathode connected by an electromotive force to adhere at least a portion of the mercury released from said biological sample to a surface of said cathode forming a mercury-alkaline metal amalgam;
(c) connecting said cathode to a reference electrode; and
(d) measuring any voltage difference between said cathode and reference electrode as a measure of the mercury level in the biological sample.

19. A device for determining mercury levels in a biological sample containing mercury, comprising a first chamber including a port for the introduction of a measured quantity of said biological sample, said first chamber including a measured quantity of an acidic solution of sufficient molarity and quantity to release at least a portion of mercury contained therein and into said acidic solution, a cathode and a first anode positioned at least partially within said acidic solution and selectively connected through an electromotive force, a solution of an alkaline metal salt and a second anode that is selectively electrically connected to said cathode through an electromotive force when said cathode is in the presence of said alkaline metal salt solution, and a reference electrode selectively electrically connected to said cathode through an indicating device.

20. The device of claim 19 wherein said source of alkaline metal salt solution is contained within a second chamber.

21. The device of claim 20 wherein said second chamber also contains said second anode and said reference electrode.

22. The device of claim 21 wherein said cathode is movable between said first and second chambers.

23. The device of claim 22 wherein said first and second chambers are joined through the use of an at least partially sealable membrane.

24. The device of claim 19 wherein said biological sample containing mercury comprises fish.

25. The device of claim 19 wherein said cathode comprises a member selected from the group consisting of aluminum, platinum, gold, silver, zinc and copper.

26. The device of claim 19 wherein said cathode comprises aluminum.

27. The device of claim 19 wherein each of said first and second anodes comprise carbon or platinum.

28. The device of claim 19 wherein said reference electrode comprises a member selected from the group consisting of carbon, platinum, gold, silver and aluminum.

29. The device of claim 19 wherein an oxidizing agent is provided within said first chamber together with said measured quantity of said acidic solution.

30. The device of claim 29 wherein said oxidizing agent comprises potassium chlorate.

31. The device of claim 19 wherein said acidic solution comprises an acid selected from the group consisting of hydrochloric acid, nitric acid and sulfuric acid.

32. The device of claim 19 wherein said alkaline metal salt comprises a member selected from the group consisting of a salt of sodium, potassium, lithium, rubidium and cesium.

33. The device of claim 32 wherein said alkaline metal salt comprises a buffered solution of sodium chloride.

34. The device of claim 19 wherein said biological sample containing mercury comprises fish and said measured quantity of acidic solution comprises a 12M hydrochloric acid solution further containing potassium chlorate as an oxidizer.

35. The device of claim 19 wherein said electromotive force between said cathode and first anode comprises a voltage between approximately 1.5 to 6 volts at a current of approximately 5 to 150 mA.

36. The device of claim 19 wherein said electromotive force between said cathode and second anode comprises a voltage between approximately 7 to 15 volts at a current of approximately 50 to 200 mA.

37. The device of claim 19 wherein said indicating device comprises a meter.

38. The device of claim 19 wherein said electromotive force comprises a regulated power supply.

39. The device of claim 19 wherein said regulated power supply comprises a constant current power supply.

40. The device of claim 19 wherein means are provided for stirring or agitating said biological sample containing mercury while said mercury contained therein is being released into said acidic solution.

41. A device for determining mercury levels in a biological sample containing mercury, comprising a first chamber including a port for the introduction of a measured quantity of said biological sample, said first chamber including a quantity of an acidic solution of sufficient molarity and quantity to release at least a portion of mercury contained therein and into said acidic solution, a cathode, an anode positioned at least partially within said acidic solution and selectively connected through an electromotive force and a solution of an alkaline metal salt and a second chamber containing a reference electrode selectively electrically connected to said cathode through an indicating device.

42. A kit for determining mercury levels in a biological sample containing mercury comprising a first chamber including a port for the introduction of a measured quantity of said biological sample and a measured quantity of an acidic solution of sufficient molarity and quantity to release at least a portion of mercury contained in said biological sample, a second chamber containing a solution of alkaline metal salt, a cathode and first anode positionable within said first chamber and a second anode and reference electrode positioned within said second chamber.

43. The kit of claim 42 wherein said cathode is movable between said first and second chambers.

44. The kit of claim 42 wherein said first and second chambers are joined through an at least partially sealable membrane.

45. The kit of claim 42 wherein said cathode comprises a member selected from the group consisting of platinum, gold, silver, zinc, copper and aluminum.

46. The kit of claim 42 wherein said first and second anodes comprise carbon or platinum.

47. The kit of claim 42 wherein said first chamber further includes an oxidizing agent.

48. The kit of claim 47 wherein said oxidizing agent comprises potassium chlorate.

49. The kit of claim 42 wherein said acid solution comprises an acid selected from the group consisting of hydrochloric acid, nitric acid and sulfuric acid.

50. The kit of claim 42 wherein said alkaline metal salt contained within said second chamber comprises a buffered solution of sodium chloride.

51. The kit of claim 42 wherein said first chamber further contains mercury ions

52. A kit for determining mercury levels in a biological sample containing mercury comprising a first chamber including a port for the introduction of a measured quantity of said biological sample, a measured quantity of an acidic solution of sufficient molarity and quantity to release at least a portion of mercury contained in said biological sample and a solution of alkaline metal salt, a cathode and anode positionable within said first chamber and a second chamber containing a reference electrode.
